(19) United States
(12) Patent Application Publication
(10) Pub. No.: US 2006/0234315 A1
(43) Pub. Date: Oct. 19, 2006

(54) Markers for sudden death in heart failure

(76) Inventors: Robert J. MacFadyen, Stourbridge (GB); Allan D. Struthers, Newport-on-Tay (GB)

Correspondence Address: STEPTOE & JOHNSON LLP, 1330 CONNECTICUT AVENUE, N.W., WASHINGTON, DC, 20036, US

(21) Appl. No.: 11/230,512

(22) Filed: Sep. 21, 2005

(30) Foreign Application Priority Data
Apr. 13, 2005 (JP) 2005-115955

(51) Int. Cl. G01N 33/567

(52) U.S. Cl. 435/7.21

(57) ABSTRACT

A method for screening, diagnosis or prognosis of sudden death in heart failure in a mammalian subject, for determining sudden death in heart failure in a mammalian subject, for identifying a mammalian subject at risk of sudden death in heart failure, or for monitoring the effect of therapy administered to a mammalian subject at experiencing imminent sudden death in heart failure included measuring the level of C-reactive protein (CRP), myeloperoxidase (MPO), neutrophil count, white blood cell count and BNP in a sample of bodily fluid from the mammalian subject. Methods for monitoring the cardiac health of a mammalian subject are further included. Kits and devices for carrying out such methods are also provided.

### CLAIM OF PRIORITY

This application claims priority under 35 U.S.C. 119(a) to Japanese Patent Application No. 2005-115955 filed Apr. 13, 2005, the entire contents of which is hereby incorporated by reference.

### TECHNICAL FIELD

This invention relates to methods and kits for diagnosing, screening or monitoring a patient for sudden death in heart failure.

### BACKGROUND

Heart failure is a chronic, progressive disease that affects 1.5-2% of the general population of the Western world. The prevalence and incidence of heart failure is growing due to an aging population. Heart failure occurs when the heart is not strong enough to pump blood efficiently around the body. Cardiovascular disease places an ever increasing burden on healthcare. In the United States alone, there are over 5 million sufferers from Congestive Heart Failure (CHF). A patient with heart failure can be a patient at high-risk of experiencing sudden death in heart failure. Sudden unexpected death in heart failure results in about 340,000 deaths each year among U.S. adults. Sudden unexpected death in heart failure, is the sudden, abrupt loss of heart function in a person who may or may not have been previously diagnosed for heart disease. The time and mode of death are unexpected. It occurs instantly or shortly after symptoms appear. The most common reason for patients to die suddenly is cardiovascular disease, in particular, coronary heart disease. Sudden unexpected death (SUD) in heart failure may also be caused by degeneration of the heart muscle, or to cardiac enlargement in patients with high blood pressure. SUD may also be caused by various heart medications that can cause arrhythmias. In particular, so-called “antiarrhythmic” drugs, even at normally prescribed doses, sometimes may produce lethal ventricular arrhythmias (“proarrhythmic” effect). Further significant changes in blood levels of potassium and magnesium (from using diuretics, for example) also can cause life-threatening arrhythmias and SUD. Other causes of SUD include electrical abnormalities due to diseases such as Wolff-Parkinson-White syndrome, blood vessel abnormalities and recreational drug use.

### SUMMARY

In general, levels of a marker in a sample from a mammalian subject, such as C-reactive protein (CRP) or neutrophil count, or white blood cell count, can be used to monitor the status of cardiac health in the mammalian subject. CRP can be used in combination with a second marker such as MPO. CRP may be used in combination with a third marker such as BNP. The status of cardiac health may be measured by screening, diagnosis or prognosis of sudden death in heart failure in a mammalian subject, or determining sudden death in heart failure, or identifying a mammalian subject at risk of sudden death in heart failure, or monitoring the effect of therapy administered to a mammalian subject experiencing imminent sudden death in heart failure. Sudden death in heart failure includes sudden unexpected death. The methods as described herein may be used in a hospital or other health-care settings or in home or other community situations. The patient can be a patient with heart failure.

In one aspect, a method for screening or diagnosing sudden death in heart failure in a mammalian subject, for determining sudden death in heart failure, for identifying a mammalian subject at risk for sudden death in heart failure or for monitoring the effect of therapy administered to a mammalian subject experiencing imminent sudden death in heart failure includes measuring a level of a first marker in a sample from the mammalian subject, wherein the first marker is C-reactive protein or neutrophil count and associating the level of the first marker with a status of cardiac health. The sample can be blood or plasma. The method can include evaluating a risk of sudden death in heart failure. The method can include assessing the level of inflammation in heart failure. Further, evaluating the risk can include comparing the level of the first marker with a threshold level of the first marker. The method of associating the level of the first marker with a status of cardiac health can include monitoring an effect of therapy administered to the subject. The method may include associating the level of the first marker with the status of cardiac health which includes comparing the level of the first marker with a level of the first marker which is indicative of the absence of the risk of sudden death in heart failure. The method may also include contacting the sample with an antibody that binds to the first marker. The antibody can be a monoclonal antibody. The method may further include measuring the level of a second marker and associating a level of the second marker with the status of cardiac health; the second marker being different from the first marker.

The method can include associating the level of the second marker with the status of cardiac health which includes evaluating a risk of sudden death in heart failure. The method can include comparing the level of the second marker with a threshold level of the second marker to evaluate the risk of sudden death in heart failure. The method can include comparing the level of the second marker with a level of the second marker which is indicative of the absence of the risk of sudden death in heart failure. The first marker can be C-reactive protein and the second marker can be a neutrophil count or white blood cell count. The neutrophil count can be obtained by standard flow cytometry. The second marker can be myeloperoxidase.

The method can include contacting the sample with an antibody that binds to the second marker. The antibody can be a monoclonal antibody. The method can further include measuring heart rate variability. Heart rate variability can be calculated by standard deviation of normal to normal intervals and triangular index. The standard deviation of normal to normal intervals can be compared with standard deviation of normal to normal intervals which are indicative of the absence of sudden death in heart failure. The triangular index can be compared with triangular index values which are indicative of the absence of sudden death in heart failure.

The method can further include measuring the level of a third marker and associating the level of the third marker with the status of cardiac health, the third marker being different from the first marker and the second marker. The method can include associating the level of the third marker with the status of cardiac health which includes evaluating a risk of sudden death in heart failure. The method can also include associating the level of the third marker with the status of cardiac health which includes comparing the level of the third marker with a level of the third marker which is indicative of the risk of death from progressive heart failure. The method can include comparing the level of the third marker with a threshold level of the third marker to evaluate the risk of sudden death in heart failure. The method can include associating the level of the third marker with the status of cardiac health includes comparing the level of the third marker with a level of the third marker which is indicative of the absence of the risk of sudden death in heart failure. The third marker can be a natriuretic peptide. The natriuretic peptide can be brain natriuretic peptide or N-terminal pro-brain natriuretic peptide.

The method can include associating the level of the third marker with the status of cardiac health which includes contacting the sample with an antibody that binds to the third marker. The antibody can be a monoclonal antibody. A kit for carrying out such methods is also provided.

In a further aspect, a method for screening or diagnosing sudden death in heart failure in a mammalian subject, for determining sudden death in heart failure, for identifying a mammalian subject at risk of sudden death in heart failure, or for monitoring the effect of therapy administered to a mammalian subject experiencing imminent sudden death in heart failure can include measuring a level of a first marker in a sample from the mammalian subject, wherein the first marker is a neutrophil count; and associating the level of the first marker with a status of cardiac health.

In a further aspect, a kit for screening, diagnosis or prognosis of sudden death in heart failure in a mammalian subject, for determining sudden death in heart failure, for identifying a mammalian subject at risk of sudden death in heart failure, or for monitoring the effect of therapy administered to a mammalian subject at experiencing imminent sudden death in heart failure can include instructions for taking a sample from said mammalian subject and associating the level of C-reactive protein with the status of cardiac health and one or more reagents for measuring the level of C-reactive protein in the sample. The sample can be blood or plasma. The kit can include instructions for associating the level of C-reactive protein with the status of cardiac health which includes evaluating the risk of sudden death in heart failure. The kit can include an antibody that binds specifically to the first marker. The antibody can be a monoclonal antibody.

The kit can include one or more reagents for measuring the level of a second marker indicative of status of cardiac health. The second marker can be myeloperoxidase. The kit can include an antibody that binds specifically to the second marker. The antibody can be a monoclonal antibody. The second marker can be neutrophil count or white blood cell count. The kit can include a device that includes a detector configured to measure and to monitor heart rate. The device can be configured to provide an output to a patient.

The kit can include one or more reagents for measuring the level of a third marker indicative of the absence of sudden death in heart failure. The levels of the third marker can be indicative of a risk of death from progressive heart failure. The third marker can be a natriuretic peptide. The natriuretic peptide can be brain natriuretic peptide or N-terminal pro-brain natriuretic peptide. The kit can include an antibody that binds specifically to the third marker. The antibody can be a monoclonal antibody.

In a further aspect, a method of monitoring the health of a mammalian subject can include measuring the level of a first marker in a sample from said mammalian subject, wherein said first marker is C-reactive protein or neutrophil count, and associating the level of the first marker with the status of cardiac health. The method can include associating the level of the first marker with the status of cardiac health which includes evaluating the risk of sudden death in heart failure. The sample can be blood or plasma. The method can include evaluating the risk which includes comparing the level of the first marker with a threshold level of the first marker. The method can include associating the level of the first marker with the status of cardiac health. This includes comparing the level of the first marker with a level of the first marker which is indicative of the absence of sudden death in heart failure. The method can include monitoring an effect of therapy administered to subject. The method can include contacting the sample with an antibody that binds to the first marker. The antibody can be a monoclonal antibody. The method can further include measuring the level of a second marker and associating a level of the second marker with the status of cardiac health; the second marker being different from the first marker. The first marker can include C-reactive protein and the second marker can include neutrophil count or white blood cell count. The second marker can include myeloperoxidase. Monitoring the health of a patient can include monitoring the risk of death or worsening condition in the patient.

The method can include evaluating a risk of sudden death in heart failure. Evaluating the risk of sudden death in heart failure can include comparing the level of the second marker with a threshold level of the second marker. The method can include contacting the sample with an antibody that binds to the second marker. The antibody can include a monoclonal antibody. The method can further include measuring heart rate variability. Heart rate variability can be calculated by standard deviation of normal to normal intervals and triangular index. The standard deviation of normal to normal intervals can be compared with standard deviation of normal to normal intervals values which are indicative of the absence of sudden death in heart failure. The triangular index can be compared with triangular index values which are indicative of the absence of sudden death in heart failure.

In a further aspect, a system for monitoring cardiac health may include a cartridge including a sample port and a first assay, wherein the first assay recognizes a first marker; and a cartridge reader including a detector configured to measure a level of the first marker recognized by the assay. The device can be configured to provide an output to a patient. The first assay can include an antibody that recognizes the first marker. The antibody can recognize C-reactive protein. The system can include a second test cartridge that includes a sample port and a second assay, wherein the second assay recognizes a second marker; the second marker being different from the first marker. The second assay can include an antibody that recognizes the second marker.

Surprisingly, CRP and neutrophil count both have incremental value in detection of SUD which coupled with measurements of heart rate variability can improve the specificity and positive prediction sufficiently to make screening for SUD in heart failure a possibility.

Many of the tests and procedures for accurately and successfully monitoring, diagnosing, managing and treating heart failure are complex, expensive and available only at a hospital or other health-care settings. Methods for patients to manage or to monitor the likelihood of heart failure at home or otherwise outside a health-care setting can be even less successful. Advantageously, sudden death can be identified or tracked by monitoring the level of CRP and/or neutrophil count in a sample from a patient.

The details of one or more embodiments are set forth in the drawings and description below. Other features, objects, and advantages will be apparent from the description, the drawings, and from the claims.

### BRIEF DESCRIPTION OF THE DRAWING

FIG. 1 is a series of graphs depicting changes in shows changes in triangular index (TI), standard deviation of all normal to normal (NN) intervals (SDNN), and ventricular extrasystole counts before death ((SUD) and progressive heart failure (PHF) groups) or the end of study.

FIG. 2 is a series of graphs depicting changes in plasma brain natriuretic peptide (BNP), C-reactive peptide (CRP), and neutrophils before either death (SUD and PHF groups) or the end of the study (for the alive group).

FIG. 3 is a graph depicting neutrophil changes in SUD patients.

FIG. 4 is a graph depicting C-reactive protein (CRP) changes in SUD patients.

FIG. 5 is a graph depicting individual standard deviations of all NN intervals (SDNN) changes in SUD patients.

FIG. 6 is a graph depicting triangular index (TI) changes in SUD patients.

FIG. 7 is a diagram illustrating a diagnostic device and an associated testing cartridge.

### DETAILED DESCRIPTION

Despite advances in the management of chronic heart failure (CHF), patients with CHF still have a high mortality rate (Ho et al., J Am Coll Cardiol. 1993:22 (Suppl):6-13; Massie et al., Curr Opin Cardiol. 1996;11 :221-6; Mcdonagh et al., Lancet 1997;350 :829-33; Cowie et al., Eur Heart J. 1997;18 :208-25). These deaths appear to occur by different modes: sudden unexpected death (SUD) and death caused by progressive heart failure (PHF) (Goldman et al., Circulation 1993;87 (Suppl):3124-33; Hinkle et al., Circulation 1982;65: 457-64). PHF deaths can be predicted by simple clinical measures such as worsening New York Heart Association (NYHA) class, lower left ventricular ejection fraction, or low exercise capacity (Szlachcic et al., Am J Cardiol. 1985;55 :1037-42; Mancini et al., Circulation 1991;83 :778-86; Roul et al., Eur Heart J. 1995; 16 :1387-98; Cohn et al., N Engl J Med. 1984;311 :819-23; Cohn et al., Circulation 1993;87 (Suppl):5-16).

In contrast, SUD in CHF is not as easy to predict. SUD is the sudden, abrupt loss of heart function in a person who may or may not have diagnosed heart disease (Nolan et al., Circulation 1998;98 :1510-6; La Rovere et al., Circulation 2003; 107 :565-70; Galiner et al., Eur Heart J. 2000;21 :475-481). SUD may occur instantly or shortly after symptoms appear. SUD is a major health problem, causing about 340,000 deaths each year among U.S. adults either before reaching a hospital or emergency room.

SUD can be triggered either by an acute coronary syndrome or by a primary arrhythmia. This means that some measure of an impending coronary event or of “arrhythmogenicity” may increase just before the SUD (La Rovere et al., Circulation 2003;107 :565-70; Galiner et al., Eur Heart J. 2000;21 :475-481).

Sudden death, also known as sudden unexpected death in congestive heart failure may often be primarily caused by an acute coronary syndrome, which is in turn due to the rupture of an “inflamed” atherosclerotic plaque (Davies et al., N Engl J Med. 1984;3 10: 1137-40). If this were true, one may expect that intraindividual signs of inflammation such as neutrophilia or C-reactive protein would increase just before the SUD. This hypothesis is made likely because of the landmark work of Ridker and colleagues (Ridker et al., N Engl J Med. 2000;342 :836-43) who showed clearly that C reactive protein is increased when measured years before the SUD. As such, there may be a possibility that a SUD event is triggered by a further, short term intraindividual increase in C-reactive protein and inflammation just before the SUD event due to the fact that sites of acute rupture in plaques have much more inflammation in them than stable plaques (van der Wal et al., Circulation 1994;89 :36-44) and by recent pathological data that plaque instability and thrombus formation exist for days to weeks before SUD (de Gouveia et al., Eur Heart J. 2002;23: 1433-40. On the other hand, if “arrhythmogenicity” contributes to SUD, then ambulatory electrocardiography may be useful, but it has so far proved variable in its ability to predict SUD (Nolan et al., Circulation 1998;98 :1510-6; La Rovere et al., Circulation 2003; 107: 565-70; Galiner et al., Eur Heart J. 2000;21 :475-481). This variability may be because all current studies are cross sectional, where one sample is taken from a large number of patients and there is then a long time gap before the SUD occurs. The alternative study design would be a longitudinal study where many repeated ECGs are taken from the same patients to look for intraindividual changes before the SUD (Nakagawa et al., Br Heart J. 1994;71 :87-8). Apart from the time gap problem, another problem with cross sectional studies is that most parameters have greater interindividual variability than they do intraindividual variability, and high interindividual variability severely limits the ability of cross sectional studies to identify individual risk. Two measures on the ambulatory electrocardiogram (ECG) that may progressively worsen as SUD approaches are heart rate variability and spontaneous ventricular extrasystole activity (Nakagawa et al., Br Heart J. 1994;71 :87-8; Bigger T. Circulation 1987;75 (Suppl):28-35). One previous report of two cases did suggest that heart rate variability worsened in patients before their SUD (Nakagawa et al., Br Heart J. 1994;71: 87-8).

A study was thus performed to see whether SUD in CHF was preceded by intraindividual increases in inflammation (C reactive protein/neutrophils) or in certain electrocardiogram (ECG) measures of arrhythmogenicity. These are the most likely “trigger” events before SUD.

A first marker indicative of sudden death in hearth failure in a mammalian subject is C-reactive protein (CRP—SwissProt P02741). A second marker indicative of sudden death in heart failure in a mammalian subject can be measured. The second marker may be measured by neutrophil count or white blood cell count. Neutrophil count or white blood cell count can be measured directly, for example, by cell counting or indirectly, for example, by measuring a level of a protein such as myeloperoxidase, or elastase. Neutrophil count may also be obtained indirectly using anti-neutrophil antibodies which can include anti-myeloperoxidase antibody. anti neutrophil elastase antibody or anti-CD11b antibody.

The second marker can be myeloperoxidase (EC 1.11.1.7) which is a major neutrophil protein. The amino acid sequence for human MPO may be found in NCBI database Accession NM_{—}000250, Accession AH_{—}002972). Other additional markers that can be measured may include markers of inflammation such as a oxidized low-density lipoprotein, a soluble adhesion molecule (e.g., E-selectin, P-selectin, intracellular adhesion molecule-i, or vascular cell adhesion molecule-1), Nourin-1, a cytokine (e.g., interleukin-1β, -6, -8, and -10 or tumor necrosis factor-alpha), or acute phase reactants (e.g., fibrinogen, or serum amyloid A (SAA)).

A third marker indicative of the absence of sudden death in heart failure in a mammalian subject may also be measured. Such markers may include natriuretic peptide, such as native atrial natriuretic peptide (ANP—see Brenner et al, Physiol. Rev., 1990, 70: 665), brain natriuretic peptide (BNP) and C-type natriuretic (CNP—see Stingo et al, Am. J. Physiol. 1992, 263: H1318), and variants or allelic variants thereof. A suitable natriuretic peptide is brain natriuretic peptide (BNP) or N-terminal pro-brain natriuretic peptide (N-BNP). Another suitable natriuretic peptide is atrial natriuretic peptide (ANP) (Hall, Eur J Heart Fail, 2001, 3:395-397).

These markers may also be used to assess the level of inflammation in heart failure in a patient. The levels of inflammation can be associated with a high risk of cardiovascular events such as sudden unexpected death (SUD), progressive heart failure (PHF), acute coronary syndrome (ACS) or stroke.

The levels of such markers may be evaluated by comparing the level of a marker with a threshold level of the marker. Such threshold levels may be set according to data pooled from large population groups such as children, young adults, the elderly, or racial/ethnic groups, or combinations thereof. The threshold levels that are measured and set for a certain population group may not be transferable or applicable from one population group to the other. Alternatively, the threshold level may also be based on threshold levels obtained from data obtained from individualized tracking. Frequent testing of a patient may provide better comparisons to the threshold levels previously established within that particular patient and would allow the physician to better assess the patient's cardiac health. The reduced intraindividual variance of marker levels would allow a more specific and individualized threshold to be defined for the patient.

The method of evaluating the levels of the markers may include observing a change in intraindividual variability, observing a change in absolute or relative level from a baseline level established within an individual or observing a positive or negative trend over time.

A level of a marker in a sample can be measured by quantifying the amount of the marker in the sample as a whole molecule, as fragments of the marker, or by measuring the marker's activity in the sample or a derivative of the sample. Fragments of the above-described markers can be measured using a fragment that will have an amino acid sequence which is unique to the marker in question. The fragment may be as few as 6 amino acids, although it may be 7, 8, 9, 10, 11, 12, 13, 14, 15 or more amino acids.

In general, an amino acid residue of the marker can be replaced by another amino acid residue in a conservative substitution. Examples of conservative substitutions include, for example, the substitution of one non-polar (i.e., hydrophobic) residue such as isoleucine, valine, leucine or methionine for another non-polar residue; the substitution of one polar (i.e. hydrophilic) residue for another polar residue, such as a substitution between arginine and lysine, between glutamine and asparagine, or between glycine and serine; the substitution of one basic residue such as lysine, arginine or histidine for another basic residue; or the substitution of one acidic residue, such as aspartic acid or glutamic acid for another acidic residue. In an conservative substitution, an amino acid residue can be replaced with an amino acid residue having a chemically similar side chain. Families of amino acid residues having side chains with chemical similarity have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A conservative substitution may also include the use of a chemically derivatized residue in place of a non-derivatized residue. A chemical derivative a residue chemically derivatized by reaction of a functional group of the residue. Examples of such chemical derivatives include, but are not limited to, those molecules in which free amino groups have been derivatized to form, for example, amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters, or other types of esters or hydrazides. Free hydroxyl groups may be derivatized to form O-acyl or O-alkyl derivatives. Also included as chemical derivatives are those polypeptides which contain one or more naturally-occurring amino acid derivatives of the twenty standard amino acids. For example, 4-hydroxyproline may be substituted for proline; 5-hydroxylsine may be substituted for histidine; homoserine may be substituted for serine; and ornithine may be substituted for lysine.

An amino acid residue of the polypeptide can be replaced by another amino acid residue in a non-conservative substitution. In some cases, a non-conservative substitution will not alter the relevant properties of the polypeptide. The relevant properties can be, without limitation, ability to bind to an antibody that recognizes CRP, MPO, N-BNP, or BNP including variants and allelic variants thereof or other biological activity.

The level of markers in a sample can be measured qualitatively or quantitatively using an assay, for example, in an immunochromatographic format. A qualitative assay can be distinguish between the presence or absence of a marker, or can distinguish between categories of marker levels in a sample, such as absent, low concentration, medium concentration or high concentration, or combinations thereof. A quantitative assay can provide a numerical measure of a marker in a sample. The assay can include contacting a marker with an antibody that recognizes that particular marker, detecting the marker by mass spectrometry, assaying a sample including cells for expression (e.g., of mRNA or polypeptide) of the marker gene by the cells, or a combination of measurements. For example, the assay can include contacting a sample with an antibody that recognizes the marker and a mass spectrometry measurement.

The markers can be detected in blood, plasma or other bodily fluids which can be obtained from a mammalian body, such as interstitial fluid, urine, whole blood, saliva, serum, lymph, gastric juices, bile, sweat, tear fluid and brain and spinal fluids. Bodily fluids may be processed (e.g. serum) or unprocessed. The mammalian subject may be a human.

The measured level of the first marker, for example CRP, may be compared with a level of CRP which is indicative of the absence of heart failure. This level may be the baseline level of CRP from one or more mammalian subjects free from SUD in heart failure, or with a previously determined reference range for CRP in such mammalian subjects. In this way, the levels can be compared with reference levels determined from population studies of subjects free from SUD in heart failure to provide a diagnosis or prognosis. Such subjects may be matched for age and/or gender. In one embodiment, the level of CRP which is indicative of risk of sudden death in heart failure may be about 3 μg/ml or more, for example, greater than 5 μg/ml, greater than 10 μg/ml, greater than 15 μg/ml, greater than 20 μg/ml, greater than 25 μg/ml or greater than 30 μg/ml from baseline values. In another embodiment, the neutrophil count which is indicative of risk of sudden death in heart failure may be about 0.5×10⁹/l, 1×10^{9/l,} 2×10⁹/l, 3×10⁹/l, 4×10⁹/l, 5×10⁹/l or more from baseline values.

In another embodiment, the level of BNP which is indicative of risk of death from progressive heart failure may be about 55 pg/ml, 65 pg/ml, 75 pg/ml, 85 pg/ml, 95 pg/ml, or 105 pg/ml from baseline values. In another embodiment, the neutrophil count which is indicative of risk of death from progressive heart failure may be about 1.5×10⁹/l, 2×10⁹/l, 2.5×10⁹/l, 3×10⁹/l or more from baseline values. In a further embodiment, the level of CRP which is indicative of risk of death in progressive heart failure may be about 5 μg/ml or more, or 10 μg/ml or more.

The precise value can vary according to assay format. To monitor the effect of therapy administered to a mammalian subject having imminent SUD, the measured level of CRP can be compared with a base level for the subject. The base level may be determined prior to commencement of the therapy. Deviations from this base level indicate whether there was an improvement or deterioration of status of cardiac health and hence whether the therapy is effective. An increased level of CRP indicates progressively imminent SUD in heart failure and vice versa. It will be appreciated by one of skill in the art that the respective levels of CRP which are for screening, diagnosis or prognosis of SUD in heart failure in a mammalian subject, for determining SUD in heart failure in a mammalian subject, for identifying a mammalian subject at risk of developing SUD in heart failure, or for monitoring the effect of therapy administered to a mammalian subject having imminent SUD in heart failure may be different from one another. These levels may be easily determined in a manner similar to that described herein.

The measured level of the second marker may be compared with a level of the second marker which is indicative of the absence of SUD in heart failure. This level may be the level of the second marker from one or more mammalian subjects free from SUD in heart failure, or with a previously determined reference range for the second marker in mammalian subjects free from SUD in heart failure. Again, the precise values can vary according to assay format.

As is explained in more detail herein, using a combination of CRP, N-BNP, neutrophil count, white blood cell count, heart rate variability can provide improved specificity and positive predictive value relative to using any of these markers alone. The result of this is that fewer echocardiographs need to be carried out to confirm heart failure, resulting in a significant cost saving. Marker levels may be provided in units of concentration, mass, moles, volume or any other measure indicating the amount of marker present.

Neutrophil count or white blood cell count may be obtained by standard flow cytometry or by microfluidic devices as described in WO2003078972, or by any other method known to a person of skill in art. The measured neutrophil count may be compared with a neutrophil count which is indicative of the absence of SUD in heart failure. This neutrophil count may be compared with the neutrophil count from one or more mammalian subjects free from SUD in heart failure, or with a previously determined reference range for the second marker in mammalian subjects free from SUD in heart failure. Heart rate variability may be measured by electrocardiogram (ECG) and standard deviation of all NN intervals (SDNN) and triangular index (TI) may be analyzed according to standard guidelines as described in Anon, Circulation 1996; 93:1043-65.

The respective levels of the first, second or third markers may be measured using an immunoassay, i.e., by contacting the sample with an antibody that binds specifically to the marker and measuring any binding that has occurred between the antibody and at least one species in the sample. Such assays may be competitive or non-competitive immunoassays. Such assays, both homogeneous and heterogeneous, are well-known in the art, wherein the analyte to be detected is caused to bind with a specific binding partner such as an antibody which has been labelled with a detectable species such as a latex or gold particle, a fluorescent moiety, an enzyme, an electrochemically active species, etc. Alternatively, the analyte may be labelled with any of the above detectable species and competed with limiting amounts of specific antibody. The presence or amount of analyte present is then determined by detection of the presence or concentration of the label. Such assays may be carried out in the conventional way using a laboratory analyser or with point of care or home testing device, such as the lateral flow immunoassay as described in EP291194.

In one embodiment, an immunoassay is performed by contacting a sample from a subject to be tested with an appropriate antibody under conditions such that immunospecific binding can occur if the marker is present, and detecting or measuring the amount of any immunospecific binding by the antibody. The antibody may be contacted with the sample for at least about 10 minutes, 30 minutes, 1 hour, 3 hours, 5 hours, 7 hours, 10 hours, 15 hours, or 1 day. Any suitable immunoassay can be used, including, without limitation, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, a marker can be detected in a fluid sample by means of a two-step sandwich assay. In the first step, a capture reagent (e.g., an anti-marker antibody) is used to capture the marker. The capture reagent can optionally be immobilised on a solid phase. In the second step, a directly or indirectly labelled detection reagent is used to detect the captured marker. In one embodiment, the detection reagent is an antibody. In another embodiment, the detection reagent is a lectin.

In one embodiment, a lateral flow immunoassay device may be used in the “sandwich” format wherein the presence of sufficient marker in a sample will cause the formation of a “sandwich” interaction at the capture zone in the lateral flow assay. The capture zone as used herein may contain capture reagents such as antibody molecules, antigens, nucleic acids, lectins, and enzymes suitable for capturing MPO and other markers described herein. The device may also incorporate one or more luminescent labels suitable for capture in the capture zone, the extent of capture being determined by the presence of analyte. Suitable labels include fluorescent labels immobilised in polystyrene microspheres. Microspheres may be coated with immunoglobulins to allow capture in the capture zone.

Other assays that may be used include, but are not limited to, flow-through devices.

In a flow-through assay, one reagent (usually an antibody) is immobilised to a defined area on a membrane surface. This membrane is then overlaid on an absorbent layer that acts as a reservoir to pump sample volume through the device. Following immobilisation, the remainder of the protein-binding sites on the membrane are blocked to minimise non-specific interactions. When the assay is used, a bodily fluid sample containing a marker specific to the antibody is added to the membrane and filters through the matrix, allowing the marker to bind to the immobilised antibody. In an optional second step (in embodiments wherein the first reactant is an antibody), a tagged secondary antibody (an enzyme conjugate, an antibody coupled to a coloured latex particle, or an antibody incorporated into a coloured colloid) may be added or released that reacts with captured marker to complete the sandwich. Alternatively, the secondary antibody can be mixed with the sample and added in a single step. If a marker is present, a coloured spot develops on the surface of the membrane.

The term “antibody” as used herein refers to immunoglobulin molecules and immunologically active portions of immunoglobulin molecules, i.e., molecules that contain an antigen binding site that specifically binds an antigen. The immunoglobulin molecules can be of any class (e.g., IgG, IgE, IgM, IgD and IgA) or subclass of immunoglobulin molecule. Antibodies includes, but are not limited to, polyclonal, monoclonal, bispecific, humanised and chimeric antibodies, single chain antibodies, Fab fragments and F(ab′)₂ fragments, fragments produced by a Fab expression library, anti-idiotypic (anti-Id) antibodies, and epitope-binding fragments of any of the above. An antibody, or generally any molecule, “binds specifically” to an antigen (or other molecule) if the antibody binds preferentially to the antigen, and, e.g., has less than about 30%, preferably 20%, 10%, or 1% cross-reactivity with another molecule. Portions of antibodies include Fv and Fv′ portions.

Antibodies for detecting CRP, MPO, BNP, neutrophils and the other markers discussed herein are available commercially. For example, anti-CRP antibodies are available from Alpha Diagnostic International, Inc., 5415 Lost Lane, San Antonio, Tex. 78238 USA and Research Diagnostics Inc Pleasant Hill Road, Flanders N.J. 07836, USA, anti-MPO and anti-BNP antibodies are available from Abcam Ltd, 21 Cambridge Science Park, Milton Road, Cambridge CB4 0TP, UK. Antibodies binding to BNP and ANP can be obtained commercially. Examples of commercially available antibodies binding to BNP are rabbit anti-human BNP polyclonal antibody (Biodesign International), rabbit anti-BNP amino acids 1-20 polyclonal antibody (Biodesign International), anti-human BNP monoclonal antibody (Immundiagnostik), and rabbit anti-human BNP amino acids 1-10 polyclonal antibody (Immundiagnostik). Examples of commercially available antibodies binding to ANP are mouse anti-human ANP monoclonal antibody (Biodesign International), rabbit anti-human ANP monoclonal antibody (Biodesign International), mouse anti-human ANP monoclonal antibody (Chemicon), rabbit anti-human ANP amino acids 95-103 antibody (Immundiagnostik), rabbit anti-human ANP amino acids 99-126 antibody (Immundiagnostik), sheep anti-human ANP amino acids 99-126 antibody (Immundiagnostik), mouse anti-human ANP amino acids 99-126 monoclonal antibody (Immundiagnostik) and rabbit anti-human a-ANP polyclonal antibody (United States Biological). Antibodies for detecting neutrophils may include anti neutrophil elastase antibody (Chemicon), anti-CD11b (C3bi receptor) antibody, anti-neutrophil antibodies.

A kit can be used to carry out the methods described herein.

Also provided is a kit for screening, diagnosis or prognosis of sudden death in heart failure in a mammalian subject, for determining sudden death in heart failure, for identifying a mammalian subject at risk of sudden death in heart failure, or for monitoring the effect of therapy administered to a mammalian subject experiencing imminent sudden death in heart failure. The kit can include instructions for taking a sample of bodily fluid from the mammalian subject, and one or more reagents for measuring the level of markers in the sample and associating the level of the markers with the status of cardiac health.

The one or more reagents may comprise an antibody that binds specifically to the first marker, as is described above. The kit may further comprise one or more reagents for measuring the level of a second marker indicative of the status of cardiac health in a mammalian subject, as is also described above. The kit may further comprise one or more reagents for measuring the level of a third marker.

The instructions for taking a sample of bodily fluid from the mammalian subject may be optional. In addition, a kit may optionally comprise one or more of the following: (1) instructions for using the kit for screening, diagnosis or prognosis of sudden death in heart failure in a mammalian subject, for determining sudden death in heart failure, for identifying a mammalian subject at risk of sudden death in heart failure, or for monitoring the effect of therapy administered to a mammalian subject experiencing imminent sudden death in heart failure; (2) a labelled binding partner to any antibody present in the kit; (3) a solid phase (such as a reagent strip) upon which any such antibody is immobilised; and (4) a label or insert indicating regulatory approval for diagnostic, prognostic or therapeutic use or any combination thereof. If no labelled binding partner to the or each antibody is provided, the or each antibody itself can be labelled with a detectable marker, e.g., a chemiluminescent, enzymatic, fluorescent, or radioactive moiety.

A device can be included in the diagnostic kit, which can optionally include one or more of the following: instructions for using the kit for event detection, diagnosis, prognosis, screening, therapeutic monitoring or any combination of these applications for the management of patients; a disposable testing cartridge containing the necessary reagents to conduct a test; or an instrument or device that measures the result of marker testing and optionally, allows manual or automatic input of other parameters, storage of the parameters, and evaluation of the parameters alongside or separate from the evaluation of the measured marker or markers.

The testing cartridge or cartridges supplied in the kit allow the user to measure as a minimum, a first marker which can be CRP, a second marker which can be myeloperoxidase or a third marker which can be a natriuretic peptide. Preferably, the testing cartridge or testing cartridges allow the sequential or serial measurement of CRP, myeloperoxidase and/or a natriuretic peptide such as BNP. A combination cartridge can test two or more different markers from a single sample.

The instrument (durable or disposable), at a minimum, measures the result of marker testing and optionally, allows manual or automatic input of other parameters, storage of said parameters, and evaluation of said parameters with or separate to the measured markers.

Referring to FIG. X, diagnostic device 100 includes display 120 and input region 140. The display 120 may be used to display images in various formats, for example, joint photographic experts group (JPEG) format, tagged image file format (TIFF), graphics interchange format (GIF), or bitmap. Display 120 can also be used to display text messages, help messages, instructions, queries, test results, and various information to patients. In some implementations, display 120 supports the hypertext markup language (HTML) format such that displayed text may include hyperlinks to additional information, images, or formatted text. Display 120 can further provide a mechanism for displaying videos stored, for example in the moving picture experts group (MPEG) format, Apple's QuickTime format, or DVD format. Display 120 can additionally include an audio source (e.g., a speaker) to produce audible instructions, sounds, music, and the like.

Input region 140 can include keys 160. In one embodiment, input region 140 can be implemented as symbols displayed on the display 120, for example when display 120 is a touch-sensitive screen. Patient instructions and queries are presented to the patient on display 120. The patient can respond to the queries via the input region.

Device 100 also includes cartridge reader 180, which accepts diagnostic test cartridges for reading. The cartridge reader 180 measures the level of a marker based on, for example, the magnitude of a color change that occurs on a test cartridge 400. Device 100 also includes probe connections 200, which connect probes (e.g., a probe of weight, temperature, heart rate, variability of heart rate, breathing rate, blood pressure, or blood oxygen saturation) to the device.

Device 100 further includes a communication port 220. Communication port 220 can be, for example, a connection to a telephone line or computer network. Device 100 can communicate the results of patient tests to a health care provider from a remote location. Likewise, the health care provider can communicate with the device 100 (e.g., to access stored test results, to adjust device parameters, or send a message to the patient).

Cartridge 400 is shown with two testing zones 420. In general, a cartridge can include 1, 2, 3, 4, or 5 or more testing zones. Each testing zone 420 can test the level of a marker. Each testing zone 420 includes a sample input 440, a control result window 460 and a test result window 480. In one embodiment, the cartridge 400 is an immunochromatographic test cartridge. Examples of immunochromatographic tests and test result readers can be found in, for example, U.S. Pat. Nos. 5,504,013; 5,622,871; 6,235,241; and 6,399,398, each of which is incorporated by reference in its entirety.

Device 100 can be used to test and record the levels of various markers that provide information about the patient's health. Various implementations of diagnostic device 100 may access programs and/or data stored on a storage medium (e.g., video cassette recorder (VCR) tape or digital video disc (DVD); compact disc (CD); or floppy disk). Additionally, various implementations may access programs and/or data accessed stored on another computer system through a communication medium including a direct cable connection, a computer network, a wireless network, a satellite network, or the like.

The software controlling the diagnostic device and providing patient feedback can be in the form of a software application running on any processing device, such as, a general-purpose computing device, a personal digital assistant (PDA), a special-purpose computing device, a laptop computer, a handheld computer, or a network appliance.

A diagnostic device may be implemented using a hardware configuration including a processor, one or more input devices, one or more output devices, a computer-readable medium, and a computer memory device. The processor may be implemented using any computer processing device, such as, a general-purpose microprocessor or an application-specific integrated circuit (ASIC). The processor can be integrated with input/output (I/O) devices to provide a mechanism to receive sensor data and/or input data and to provide a mechanism to display or otherwise output queries and results to a service technician. Input device may include, for example, one or more of the following: a mouse, a keyboard, a touch-screen display, a button, a sensor, and a counter.

The display 120 may be implemented using any output technology, including a liquid crystal display (LCD), a television, a printer, and a light emitting diode (LED). The computer-readable medium provides a mechanism for storing programs and data either on a fixed or removable medium. The computer-readable medium may be implemented using a conventional computer hard drive, or other removable medium such as those described above with reference to. Finally, the system uses a computer memory device, such as a random access memory (RAM), to assist in operating the diagnostic device.

The device 100 can provide access to applications such as a medical records database or other systems used in the care of patients. In one example, the device connects to a medical records database via communication port 220. Device 100 may also have the ability to go online, integrating existing databases and linking other websites. Online access may also provide remote, online access by patients to medical information, and by caregivers to up-to-date test results reflecting the health of patients. The device can be used in a hospital, physician's office, clinic, and patient's home either by the patient or an attendant care giver.

The device can be configured to respond to the measured level of a marker, in particular when the level of the marker indicates a change in the patient's health status. For example, the device can be configured to store the results of tests and determine changes in the levels of markers over time. A change in results over time can be an acute change or a chronic change. An acute change can be a significant change in the level of a marker over a short period of time. The magnitude of change and period of time can be different for each marker. The device can be configured to compare each new test result either to a stored values of recent test results (e.g., the previous 1, 2, 3, 4, 5 or more results), or to an aggregate measure of recent test results (such as an average) to determine if an acute change has occurred. In one example, an acute change is detected by the percentage change in a test result from the previous result.

Chronic changes can be detected as well. A chronic change can be a change in the level of a marker that occurs over a long period of time. For example, a chronic change can occur such that many testing intervals pass without an acute change being detected, yet the level of marker is significantly different. To detect a chronic change, the device can compare the results of each new test to a stored result of an earlier test, or to an aggregate measure of earlier tests. For detecting chronic changes, the earlier test can be, for example, 4-12 weeks prior to the new test result. In one example, the aggregate measure can be a rolling average, such as a 4-week, 8-week, or 12-week rolling average.

The device can also be configured to compare test results to a stored threshold value or range. The threshold value can be an upper or lower limit or range of values. Thus, the device can determine if the measured value of a marker, or group of markers, is a safe level, a dangerous level, or indicates an emergency. The device can alert the patient to the results of the test and can be configured, when appropriate to instruct the patient to seek medical care.

The device can also be configured to track combinations of markers, for example, an average value of two markers, the difference in level between two markers, a ratio of the levels of two markers, or whether two or more markers exceed their respective threshold values at the same time. The device can be configured to track one or more markers in combination with a patient's signs and symptoms.

The device can be personalized for a patient. The threshold values and other parameters for each marker can be adjusted (for example, by a physician or other caregiver) based on the circumstances of the patient, such as, for example, age, gender, or disease status. The questions and responses that the device presents to the patient can also be adjusted.

Examples of implementing the method follow.

### EXAMPLES

Study Group and Data Collection

34 patients who met the following criteria: CHF for ≧18 months, clinical stability for three months; NYHA class II to IV; CHF caused by coronary artery disease (94%) or idiopathic dilated cardiomyopathy; impaired left ventricular rejection fraction of <40%; no acute coronary event within six months; and sinus rhythm were recruited for this study. Exclusion criteria were planned coronary revascularisation; chronic obstructive pulmonary disease; significant renal dysfunction; diabetes mellitus; autonomic neuropathy; limiting peripheral arterial disease; implanted pacemaker; treatment with b blockers; and atrial fibrillation. The local Tayside ethics and research committee approved the study before recruitment. All patients gave written and informed consent to participate in the study.

Patients were seen at monthly intervals in their own home. A 24 hour ambulatory ECG monitor (Tracker2, Reynolds Medical Ltd, Hertford, UK) was applied and calibrated. Blood samples were taken after ≧20 minutes supine rest. The 24 hour ECG results were processed with a Reynolds pathfinder 600 series workstation. Neutrophils were counted by our routine haematology laboratory by standard flow cytometry. Stored plasma was used for high sensitivity C reactive protein analysis with a standard commercial enzyme linked immunosorbent assay (ELISA) kit (Immuno-biological Laboratories, Hamburg, Germany). Chlamydia pneumoniae antibodies (IgG, IgA, IgM) and immune complexes were analyzed on all samples as described before (Tavendale et al., Eur Heart J. 2002;23:301-7). All monthly samples were analyzed for neutrophils, brain natriuretic peptide (BNP), and aldosterone. C reactive protein and chlamydiae were measured only on a single baseline sample and in the last three samples for each patient before death or at the end of study.

All tapes were subjected to standard Holter analysis with artefacts confirmed manually. Subsequently, time domain heart rate variability was analyzed automatically from RR intervals that had normal morphology and cycle lengths between 80-120% of the preceding cycle duration. All tapes were analyzed and their data files were reviewed and edited by an investigator (AMAS) blinded to the individual patient's clinical status. On average, 97% of the data for each patient were available for further analysis after editing. Death was classified as PHF or SUD based on the conventional expert committee view that SUD was a death within one hour of new symptoms without evidence of PHF before the new symptoms began (Narang et al. Eur Heart J. 1996;17:1390-403).

Heart rate variability was analyzed according to standard guidelines (Anon. Circulation 1996; 93:1043-65).

The analyses were done with SPSS (version 10, SPSS Inc, Chicago, Ill., USA). Most of the data were normally distributed. Data that were not normally distributed or were borderline in terms of normal distribution were analyzed as they were and after a log transformation. Mean values at different time points were compared by repeated measurements analysis of variance and linear contrasts and when appropriate a one way analysis of variance followed with post hoc Bonferroni correction. Values of p<0.05 were accepted as significant. The coefficients of variation of neutrophils, aldosterone, C reactive protein, BNP, standard deviation of all NN intervals (SDNN), and triangular index are 0.8%, 6%, 13%, 15%, 0.3%, and 1.2%, respectively.

No patient was treated with B blockade, as was standard practice at the time (mid 1990s) (Table 1). The mean (SD) follow up period was 20 (5) months. Nine patients died during follow up, four of PHF (during months 7, 12, 21, and 24) and five of SUD (during months 6, 11, 15, 20, and 21). It is worth noting that all PHF deaths occurred within hospital and all SUD occurred in the community, which helps to validate that the deaths were correctly classified (Narang et al. Eur Heart J. 1996; 17:1 390-403).

TABLE 1
Clinical characteristics of study subjects and baseline treatment

| | Mean (SEM) or number |
|---|---|
| Parameter total number | |
| Age (years) | 69 (1.3) |
| Diastolic blood pressure (mm Hg) | 72 (2.2) |
| Systolic blood pressure (mm Hg) | 126 (3.7) |
| Heart rate (beats/min) | 72 (2.2) |
| 24 hour SDANN | 142 (7.2) |
| 24 hour SDNNi | 70 (8.1) |
| Weight (kg) | 77 (2) |
| Body mass index | 30 (0.8) |
| Plasma urea (mmol/l) | 7 (0.8) |
| Plasma creatinine (mmol/l) | 131 (6.8) |
| Plasma sodium (mmol/l) | 136 (0.3) |
| Plasma ACE activity(U/l) | 12 (3.4) |
| NYHA class II/III/IV | 20/11/3 |
| LVEF (%) | 29 (1.3) |
| Previous myocardial infarction | 32 |
| Dilated cardiomyopathy* | 2 |
| Baseline treatment | |
| Furosemide (frusemide) | 29 |
| ACE inhibitor | 29 |
| Nitrate | 14 |
| Digoxin | 12 |
| Aspirin | 22 |

*Based on global left ventricular dilatation and lack of symptoms or signs of ischaemic heart disease at any time.
ACE, angiotensin converting enzyme;
LVEF, left ventricular ejection fraction;
NYHA, New York Heart Association;
SDANN, average of NN intervals for all five minute segments;
SDNNi, mean (SD) of NN intervals for all five minute segments.

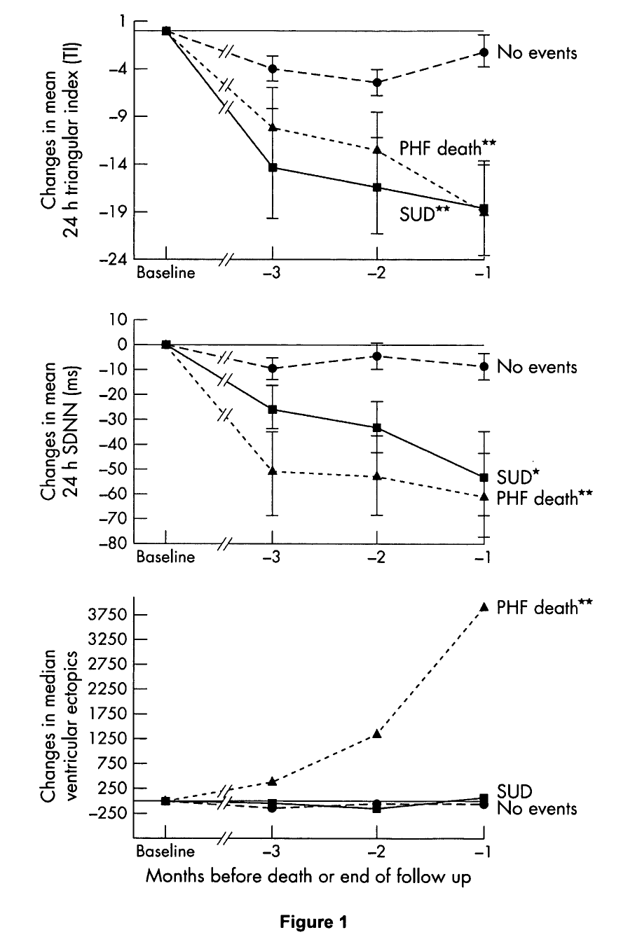

There were no significant differences at baseline except that C reactive protein and non-sustained ventricular tachycardia (NSVT) events were higher in the PHF group (Table 2). On the other hand, intraindividual changes in several parameters were highly significantly different between the three groups. FIG. 1 show changes (mean (SEM)) from baseline to the last three measurements (triangular index (TI)), standard deviation of all NN intervals (SDNN), and ventricular extrasystole counts) before either death ((SUD) and progressive heart failure (PHF) groups) or the end of the study (for the alive group). // indicates multiple measurements. *p<0.05; **p<0.01.). The patients who died (SUD and PHF) had significant intraindividual worsening in the sequential changes between baseline and the last three values of mean 24 hour heart rate variability (triangular index and SDNN) in contrast to the control survival group (see FIG. 1).

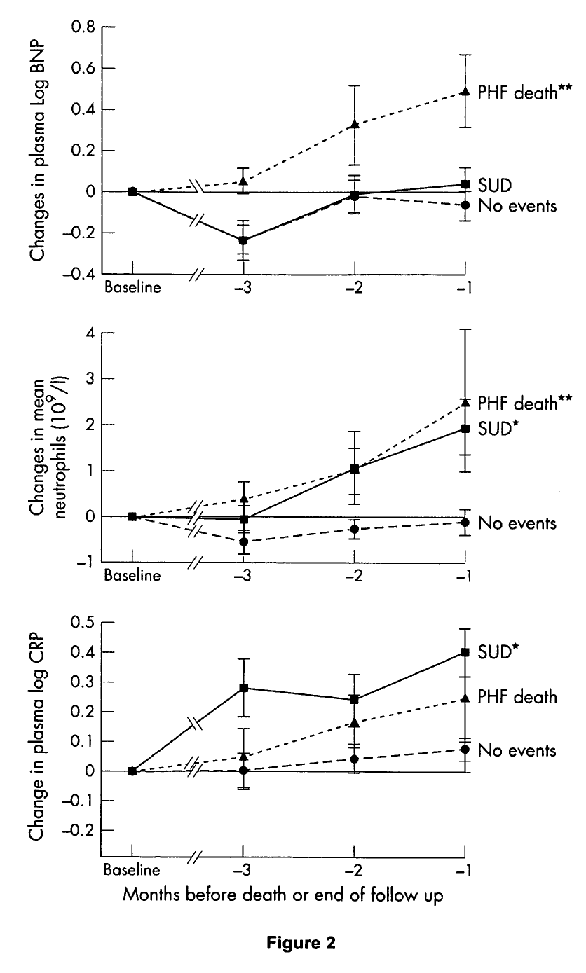

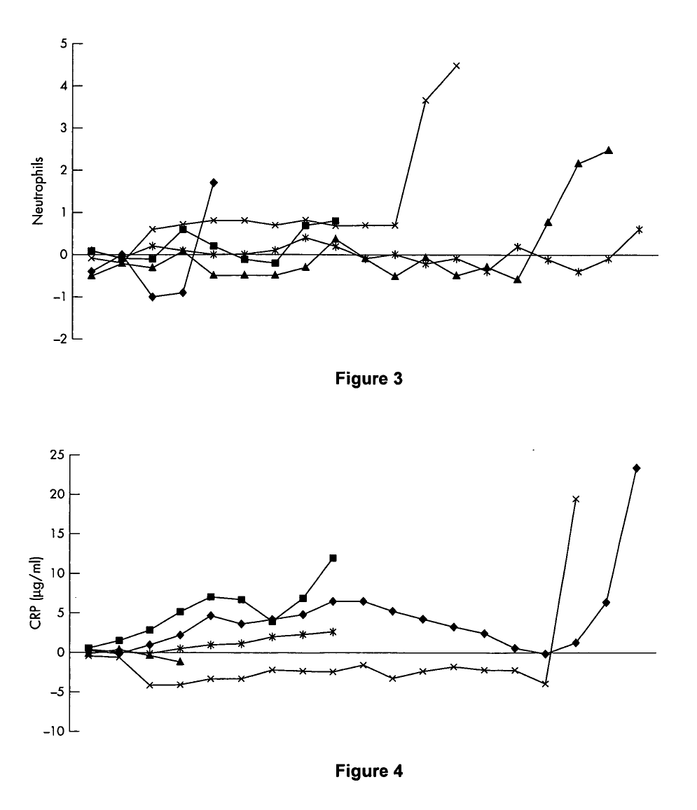

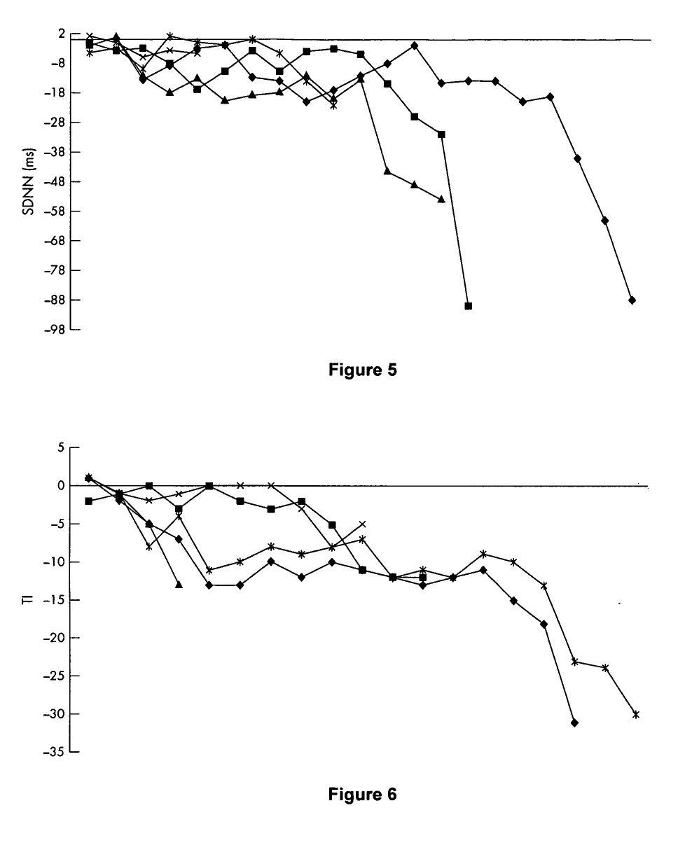

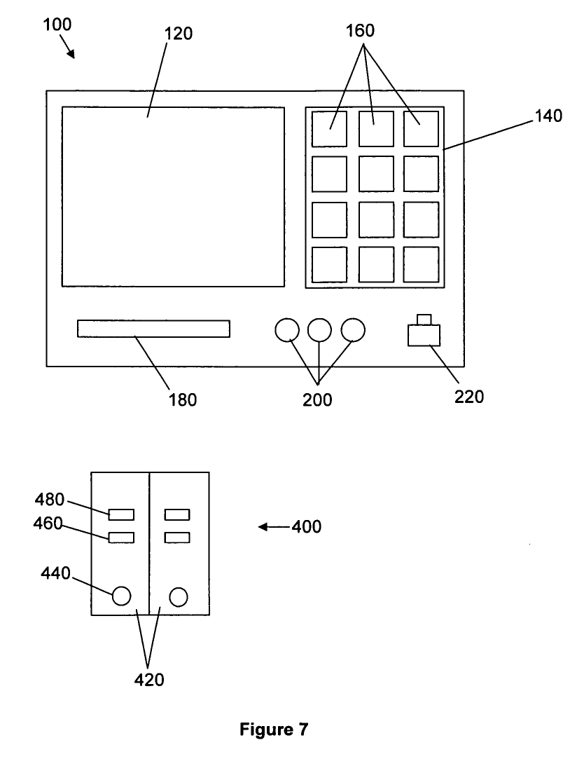

FIG. 2 shows changes (mean (SEM)) from baseline to the last three measurements of plasma brain natriuretic peptide (BNP), C-reactive peptide (CRP), and neutrophils before either death (SUD and PHF groups) or the end of the study (for the alive group). // indicates multiple measurements. *p<0.05; **p<0.01. A similar pattern was seen for the white cell count, the neutrophil count, and C reactive protein in that they all increased progressively before death in both the PHF group and the SUD group (see FIG. 2). FIGS. 3, 4, 5, and 6 show the individual data of those who died suddenly. Specifically, FIG. 3 shows individual neutrophil changes from baseline until SUD in the five SUD patients. Each symbol represents a monthly value in each patient sequentially before their SUD. FIG. 4 shows individual C-reactive protein (CRP) changes from baseline until SUD in the five SUD patients. Each symbol represents a monthly value in each patient sequentially before their SUD. FIG. 5 shows individual standard deviations of all NN intervals (SDNN) changes from baseline until SUD in the five SUD patients. Each symbol represents a monthly value in each patient sequentially before their SUD. FIG. 6 shows individual triangular index (TI) changes from baseline until SUD in the five SUD patients. Each symbol represents a monthly value in each patient sequentially before their SUD.

TABLE 2
Baseline values and differences in means for the last three measurements
compared with baseline values in various parameters

| | | | | p Value | p Value |
|---|---|---|---|---|---|
| Parameters | | | | (alive v PHF) | (alive v SUD) |
| | Alive | PHF | SUD | | |
| Single baseline value | (n = 25) | (n = 4) | (n = 5) | | |
| LVEF (%) | 29 (1.2) | 26 (5.5) | 27 (4) | 0.8 | 0.7 |
| Furosemide (mg/day) | 61 (10.8) | 56 (17.5) | 40 (14) | 0.8 | 0.3 |
| Triangular index | 38 (2.6) | 31 (6) | 46 (5.6) | 0.34 | 0.24 |
| SDNN (ms) | 142 (9) | 114 (17) | 173 (20) | 0.26 | 0.16 |
| MHR (beats/min) | 70.6 (2.9) | 64 (4.8) | 70.8 (5.3) | 0.4 | 0.98 |
| Ventricular extrasystoles | 1606 (356) | 2116 (1267) | 1008 (427) | 0.63 | 0.96 |
| NSVT events/patient | 0.4 (0.12) | 4.7 (2.8) | 0.2 (0.17) | 0.001 | 0.8 |
| Plasma potassium (mmol/l) | 5.01 (0.7) | 5.4 (0.3) | 5.06 (0.8) | 0.27 | 0.89 |
| Plasma creatinine (mmol/l) | 127 (8) | 145 (28) | 134 (17) | 0.48 | 0.71 |
| Plasma BNP (pg/ml) | 61 (9.9) | 48 (26) | 93 (20.9) | 0.38 | 0.14 |
| Plasma aldosterone (pg/ml) | 142 (26) | 102 (45) | 271 (116) | 0.68 | 0.22 |
| Plasma WBC (10⁹/l) | 6.9 (0.42) | 7.9 (0.49) | 5.9 (0.49) | 0.25 | 0.32 |
| Plasma neutrophil (10⁹/l) | 4.1 (0.3) | 5.6 (0.8) | 3.7 (0.79) | 0.07 | 0.59 |
| C reactive protein (mg/ml) | 4.2 (0.6) | 13 (5) | 7 (3.9) | 0.02 | 0.72 |

| | | | | |
|---|---|---|---|---|
| | Intraindividual changes | Intraindividual changes | | |
| | from baseline between | from baseline between | | |
| Last three measures | PHF and alive patients | SUD and alive patients | | |
| 24 hour triangular index | 211.4 (3.9) | 211.3 (3.5) | 0.023 | 0.01 |
| 24 hour SDNN (ms) | 251 (13.4) | 236.8 (11.8) | 0.002 | 0.013 |
| 24 hour MHR (beats/min) | +14 (5.6) | 24.4 (4.9) | 0.04 | 0.25 |
| 24 hour ventricular | +6293 (964) | +812 (850) | 0.0001 | 0.225 |
| extrasystoles | | | | |
| 24 hour log ventricular | +1 (0.36) | +0.17 (0.3) | 0.028 | 0.59 |
| extrasystoles | | | | |
| NSVT events/patient | +48 (14) | +0.04 (12) | 0.002 | 0.99 |
| Plasma BNP (pg/ml) | +81.6 (22) | 213.5 (19.5) | 0.003 | 0.49 |
| Log plasma BNP | +0.45 (0.17) | 20.03 (0.15) | 0.044 | 0.84 |
| Plasma aldosterone (pg/ml) | +183 (50) | 7.7 (44) | 0.003 | 0.86 |
| Log plasma aldosterone | +0.43 (0.12) | +0.02 (0.11) | 0.005 | 0.82 |
| Plasma creatinine (mmol/l) | 21.8 (16) | 211 (13) | 0.339 | 0.91 |
| Plasma neutrophil (10⁹/l) | +2.3 (0.62) | +1.5 (0.55) | 0.003 | 0.026 |
| C reactive protein (mg/ml)* | +9 (2.6) | +5 (2.3) | 0.001 | 0.04 |
| Log C reactive protein* | +0.11 (0.13) | +0.26 (0.12) | 0.39 | 0.039 |

Data are mean (SEM). Some data are borderline between being normally distributed and not being normally distributed. For those parameters, the data are presented and analyzed in two ways (logarithmically transformed and not transformed).
*Only one baseline value available for C reactive protein.
BNP, brain natriuretic peptide;
MHR, maximum heart rate;
NSVT, non-sustained ventricular tachycardia;
PHF, progressive heart failure;
SDNN, standard deviation of all
NN intervals;
SUD, sudden unexpected death;
WBC, white blood cells.

A clear change was seen in most cases before SUD, which was not seen in the alive group as judged by the data in the lower part of Table 2. Unlike others, BNP was not found to predict sudden death in CHF. BNP however was found to be predictive of death in progressive heart failure.

Patients who died in hospital of PHF also had significant increases in 24 hour mean ventricular extrasystoles, NSVT events, and 24 hour mean maximum heart rate in contrast to both SUD and control groups. In addition several parameters, which usually detect a worsening clinical state, followed the same pattern. These were 24 hour mean maximum heart rate, BNP, and aldosterone. All of these parameters behaved as expected in that they increased progressively in those who died of PHF but were unchanged in the SUD group. No significant changes were noted in any of the C pneumoniae titres or in immune complexes.

The problem being addressed here is that, as stated in an editorial in Circulation, “we currently lack the tools to identify most people at risk of a sudden cardiac arrest with a degree of accuracy sufficient to warrant major therapeutic interventions” (Zipes. Circulation 2001;104:2506-8).

In particular, SUD is preceded by progressive intraindividual decreases in heart rate variability and intraindividual increases in markers of inflammation (neutrophils and C reactive protein), but not by any change in the traditional measures of disease progression such as BNP, aldosterone, heart rate, and extrasystoles. Indeed, the diagnostic accuracy of the changes in C reactive protein, neutrophils, and heart rate variability was fairly good with sensitivities of 60-100% and specificities of 84-92%, which meant that such changes were very seldom seen in the alive group. On the other hand, PHF deaths but not SUDs were preceded by intraindividual worsening not only in heart rate variability and inflammation but also by worsening in the traditional measures of disease severity: BNP, aldosterone, extrasystoles, and NSVT. No evidence was found to specifically implicate C pneumoniae in the intraindividual increases in inflammation.

The correct classification of those who died into PHF and SUD groups is clearly important. That all PHF deaths occurred during hospitalization while all SUDs occurred in the community is some indication that our classification is correct. This can also be supported by the substantial increase in conventional measures of disease severity in the PHF death group but not in the SUD group. Our heart rate variability data support the recent large UK-HEART (UK heart failure evaluation and assessment of risk trial), where low SDNN predicted death caused by PHF (Nolan et al., Circulation 1998;98: 1510-6). However, in contrast to our study, in UK-HEART no particular abnormality in heart rate variability was seen in the SUD group; however, UK-HEART was a cross sectional study where the time gap between the heart rate variability measure and the subsequent SUD may have been too long to detect such a relation. This may explain why a link between heart rate variability and SUD was more detectable in the present longitudinal study with frequent heart rate variability monitoring than in UK-HEART.

A key question is whether intraindividual increases in “inflammation” and intraindividual worsening in heart rate variability may be related. New information suggests that this is possible. Inflammation produces endothelial dysfunction, (Bhagat et al., Cardiovasc Res. 1996;53:481-2; Bhagat et al., Circulation 1997;96:3042-7) which in turn predicts coronary events ( Al Suwaidi et al., Circulation 2000; 101 :948-54; Schachinger et al., Circulation 2000;101:1899-906). Endothelial dysfunction is also associated with reduced vascular nitric oxide, which is known to worsen autonomic function as indicated by heart rate variability (Chowdhary et al., Clin Sci. 1999;97:5-17; Spieker et al., J Am Coll Cardiol. 2000;36:213-8). The final link in this hypothesis is that autonomic (vagal) dysfunction is probably one of many arrhythmogenic stimuli (Zuanetti et al., Circ Res. 1987;61 :429-35). Recent data are generally consistent with this in that SUD in CHF is often but not always associated with an identifiable acute coronary event at necropsy (Uretsky et al., Circulation 2000;102:611-6) which may be preceded by inflammation induced endothelial dysfunction.

These results have implications for risk stratification of patients with CHF, since impending SUD may be identifiable by sequential measuring of heart rate variability, neutrophils, or C reactive protein. Impending PHF death is usually easy to recognize at the bedside, whereas impending SUD is difficult to predict at the bedside. This means that it should be relatively easy clinically to ascertain whether an intraindividual increase in inflammation or autonomic dysfunction is heralding a SUD or a PHF death. A measure of heart rate or neurohormones may assist this differentiation, if doubt still exists. This may lead to better targeting of invasive procedures such as coronary revascularisation, insertion of coated stents or implantable defibrillators, or even transplantation towards those at high risk of imminent SUD. These measures may also help in the selection of patients for targeted anti-inflammatory treatments to prevent SUD. This may even involve short term administration of fairly “toxic” anti-inflammatories such as cyclosporine or steroids, which would be warranted if the patient is identified to be at risk of an imminent SUD but not warranted if the SUD risk is distant. Indeed the IMPRESS (immunosuppressive therapy for the prevention of restenosis after coronary artery stent implantation) study showed that short term high dose prednisolone can reduce cardiovascular events dramatically in a similar but different population (Versaci et al., J Am Coll Cardiol. 2002;40:1935-42). The ventricular extrasystole data are interesting in that worsening ventricular extrasystoles and increased NSVT occur only before PHF deaths but not before SUDs. This supports a previous review article that suggested that ventricular extrasystoles are a consequence of disease progression rather than a specific cause of future sudden death (Packer, Circulation 1992;85:50-6). The number of NSVT events and the number of patients in the SUD group are both small, which makes these NSVT results unreliable.

It is worth commenting that this study was done before the use of β blockers and implantable cardioverter-defibrillators in CHF, although both should defer deaths rather than prevent them entirely. The same intraindividual changes can still occur before the deferred death. In summary, the data raise the possibility that SUD may be predictable from intraindividual changes in neutrophils, C reactive protein, or heart rate variability rather than using absolute across the board interindividual values. This was a relatively small pilot study, although the work involved was large—540 ambulatory ECG tapes and 540 assays of most analytes. However, the credibility of our results is increased by the finding that changes were the same in two independent measures of inflammation (C reactive protein and neutrophils). Nevertheless, larger studies will be needed to confirm this intriguing observation. Monthly neutrophil counts may ultimately prove to be a cheap and effective way of targeting novel treatments to patients with CHF at high risk of impending SUD (Miller et al., J Heart Lung Transplant 1995;14:1143-53). Furthermore, these data may be relevant to a wider group of patients, since half of all SUDs are said to occur in patients with at least a history of CHF (Hinkle et al., Circulation 1982;65:457-64).

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

Each and every reference cited herein is hereby incorporated in its entirety for all purposes to the same extent as if each reference were individually incorporated by reference. Furthermore, while the invention has been described in detail with reference to preferred embodiments thereof, it will be apparent to one skilled in the art that various changes can be made, and equivalents employed, without departing from the scope of the invention.

What is claimed is:

1. A method for screening or diagnosing sudden death in heart failure in a mammalian subject, for determining sudden death in heart failure, for identifying a mammalian subject at risk for sudden death in heart failure or for monitoring the effect of therapy administered to a mammalian subject experiencing imminent sudden death in heart failure, said method comprising:
measuring a level of a first marker in a sample from the mammalian subject, wherein the first marker is C-reactive protein or neutrophil count; and
associating the level of the first marker with a status of cardiac health.

2. The method of claim 1, wherein the sample is blood or plasma.

3. The method of claim 1, wherein associating the level of the first marker with the status of cardiac health includes evaluating a risk of sudden death in heart failure.

4. The method of claim 1, wherein associating the level of the first marker with the status of cardiac health includes assessing the level of inflammation in heart failure.

5. The method of claim 3, wherein evaluating the risk includes comparing the level of the first marker with a threshold level of the first marker.

6. The method of claim 1, wherein associating the level of the first marker with the status of cardiac health includes monitoring an effect of therapy administered to the subject.

7. The method of claim 1, wherein associating the level of the first marker with the status of cardiac health includes comparing the level of the first marker with a level of the first marker which is indicative of the absence of the risk of sudden death in heart failure.

8. The method of claim 1, wherein associating the level of the first marker with the status of cardiac health includes contacting the sample with an antibody that binds to the first marker.

9. The method of claim 8, wherein the antibody is a monoclonal antibody.

10. The method of claim 1, further comprising measuring the level of a second marker and associating a level of the second marker with the status of cardiac health; the second marker being different from the first marker.

11. The method of claim 10, wherein associating the level of the second marker with the status of cardiac health includes evaluating a risk of sudden death in heart failure.

12. The method of claim 11, wherein evaluating the risk of sudden death in heart failure includes comparing the level of the second marker with a threshold level of the second marker.

13. The method of claim 10, wherein associating the level of the second marker with the status of cardiac health includes comparing the level of the second marker with a level of the second marker which is indicative of the absence of the risk of sudden death in heart failure.

14. The method of claim 10, wherein the first marker is C-reactive protein and the second marker is a neutrophil count or white blood cell count.

15. The method of claim 14, wherein the neutrophil count is obtained by standard flow cytometry.

16. The method of claim 10, wherein the second marker is myeloperoxidase.

17. The method of claim 10, wherein associating the level of the second marker with the status of cardiac health includes contacting the sample with an antibody that binds to the second marker.

18. The method of claim 17, wherein the antibody is a monoclonal antibody.

19. The method of claim 1, further comprising measuring heart rate variability.

20. The method of claim 19, wherein heart rate variability is calculated by standard deviation of normal to normal intervals and triangular index.

21. The method of claim 20, wherein the standard deviation of normal to normal intervals are compared with standard deviation of normal to normal intervals which are indicative of the absence of sudden death in heart failure.

22. The method of claim 21, where triangular index is compared with triangular index values which are indicative of the absence of sudden death in heart failure.

23. The method of claim 1, further comprising measuring the level of a third marker and associating the level of the third marker with the status of cardiac health, the third marker being different from the first marker and the second marker.

24. The method of claim 23, wherein associating the level of the third marker with the status of cardiac health includes evaluating a risk of sudden death in heart failure.

25. The method of claim 24, wherein evaluating the risk of sudden death in heart failure includes comparing the level of the third marker with a threshold level of the third marker.

26. The method of claim 23, wherein associating the level of the third marker with the status of cardiac health includes comparing the level of the third marker with a level of the third marker which is indicative of the absence of the risk of sudden death in heart failure.

27. The method of claim 23, wherein associating the level of the third marker with the status of cardiac health includes comparing the level of the third marker with a level of the third marker which is indicative of the risk of death from progressive heart failure.

28. The method of claim 23, wherein the third marker is a natriuretic peptide.

29. The method of claim 28, wherein the natriuretic peptide is brain natriuretic peptide or N-terminal pro-brain natriuretic peptide.

30. The method of claim 23, wherein associating the level of the third marker with the status of cardiac health includes contacting the sample with an antibody that binds to the third marker.

31. The method of claim 30, wherein the antibody is a monoclonal antibody.

32. A kit for carrying out the method of claim 1.

33. A method for screening or diagnosing sudden death in heart failure in a mammalian subject, for determining sudden death in heart failure, for identifying a mammalian subject at risk of sudden death in heart failure, or for monitoring the effect of therapy administered to a mammalian subject experiencing imminent sudden death in heart failure, said method comprising
measuring a level of a first marker in a sample from the mammalian subject, wherein the first marker is a neutrophil count; and
associating the level of the first marker with a status of cardiac health.

34. A kit for screening, diagnosis or prognosis of sudden death in heart failure in a mammalian subject, for determining sudden death in heart failure, for identifying a mammalian subject at risk of sudden death in heart failure, or for monitoring the effect of therapy administered to a mammalian subject at experiencing imminent sudden death in heart failure, the kit comprising:
instructions for taking a sample from said mammalian subject and associating the level of C-reactive protein with the status of cardiac health;
one or more reagents for measuring the level of C-reactive protein in the sample.

35. The kit of claim 34, wherein associating the level of C-reactive protein with the status of cardiac health includes evaluating the risk of sudden death in heart failure.

36. The kit of claim 34, wherein the sample is blood or plasma.

37. The kit of claim 34, wherein the one or more reagents comprise an antibody that binds specifically to the first marker.

38. The kit of claim 34, wherein the antibody is a monoclonal antibody.

39. The kit of claim 34, further comprising one or more reagents for measuring the level of a second marker indicative of status of cardiac health.

40. The kit of claim 34, wherein the second marker is myeloperoxidase.

41. The kit of claim 39, wherein the one or more reagents for measuring the second marker comprises an antibody that binds specifically to the second marker.

42. The kit of claim 41, wherein the antibody is a monoclonal antibody.

43. The kit of claim 39, wherein the second marker is neutrophil count or white blood cell count.

44. The kit of claim 34, further comprising a device including a detector configured to measure and to monitor heart rate.

45. The kit of claim 44, wherein the device is configured to provide an output to a patient.

46. The kit of claim 34, further comprising one or more reagents for measuring the level of a third marker indicative of the absence of sudden death in heart failure.

47. The kit of claim 46, wherein the levels of the third marker is indicative of a risk of death from progressive heart failure.

48. The kit of claim 46, wherein the third marker is a natriuretic peptide.

49. The kit of claim 46, wherein the natriuretic peptide is brain natriuretic peptide or N-terminal pro-brain natriuretic peptide.

50. The kit of claim 46, wherein the one or more reagents for measuring the third marker comprises an antibody that binds specifically to the third marker.

51. The kit of claim 50, wherein the antibody is a monoclonal antibody.

52. A method of monitoring the health of a mammalian subject comprising measuring the level of a first marker in a sample from said mammalian subject, wherein said first marker is C-reactive protein or neutrophil count, and associating the level of the first marker with the status of cardiac health.

53. The method of claim 52, wherein associating the level of the first marker with the status of cardiac health includes evaluating the risk of sudden death in heart failure.

54. The method of claim 52, wherein the sample is blood or plasma.

55. The method of claim 53, wherein evaluating the risk includes comparing the level of the first marker with a threshold level of the first marker.

56. The method of claim 52, wherein associating the level of the first marker with the status of cardiac health includes comparing the level of the first marker with a level of the first marker which is indicative of the absence of sudden death in heart failure.

57. The method of claim 52, wherein associating the level of the first marker with the status of cardiac health includes monitoring an effect of therapy administered to subject.

58. The method of claim 52, wherein associating the level of the first marker with the status of cardiac health includes contacting the sample with an antibody that binds to the first marker.

59. The method of claim 58, wherein the antibody is a monoclonal antibody.

60. The method of claim 52, further comprising measuring the level of a second marker and associating a level of the second marker with the status of cardiac health; the second marker being different from the first marker.

61. The method of claim 60, wherein the first marker is C-reactive protein and the second marker is neutrophil count or white blood cell count.

62. The method of claim 60, wherein the second marker is myeloperoxidase.

63. The method of claim 60, wherein associating the level of the second marker with the status of cardiac health includes evaluating a risk of sudden death in heart failure.

64. The method of claim 63, wherein evaluating the risk of sudden death in heart failure includes comparing the level of the second marker with a threshold level of the second marker.

65. The method of claim 60, wherein associating the level of the second marker with the status of cardiac health includes contacting the sample with an antibody that binds to the second marker.

66. The method of claim 65, wherein the antibody is a monoclonal antibody.

67. The method of claim 52, further comprising measuring heart rate variability.

68. The method of claim 67, wherein heart rate variability is calculated by standard deviation of normal to normal intervals and triangular index.

69. The method of claim 68, wherein the standard deviation of normal to normal intervals are compared with standard deviation of normal to normal intervals values which are indicative of the absence of sudden death in heart failure.

70. The method of claim 68, where triangular index is compared with triangular index values which are indicative of the absence of sudden death in heart failure.

71. A system for monitoring cardiac health, comprising:
a cartridge including a sample port and a first assay, wherein the first assay recognizes a first marker; and
a cartridge reader including a detector configured to measure a level of the first marker recognized by the assay.

72. The system of claim 71, wherein device is configured to provide an output to a patient.

73. The system of claim 71, wherein the first assay includes an antibody that recognizes the first marker.

74. The system of claim 71, wherein the antibody recognizes C-reactive protein.

75. The system of claim 71, further comprising a second test cartridge including a sample port and a second assay, wherein the second assay recognizes a second marker; the second marker being different from the first marker.

76. The system of claim 75, wherein the second assay includes an antibody that recognizes the second marker.
